(19) United States
(12) Patent Application Publication
(10) Pub. No.: US 2006/0228339 A1
(43) Pub. Date: Oct. 12, 2006

(54) Methods of preparing transplantable product for treatment of skin defects

(76) Inventor: Zheng-Pin Wang, Ellicott City, MD (US)

Correspondence Address: Jerome J Norris, Suite 305, 1901 Pennsylvania Avenue NW, Washington, DC, 20006, US

(21) Appl. No.: 10/564,740

(22) PCT Filed: Aug. 22, 2003
(86) PCT No.: PCT/US03/26252

§ 371 Date: Jan. 19, 2006

Related U.S. Application Data
(60) Provisional application No. 60/478,427, filed on Jun. 16, 2003.

(51) Int. Cl. A61K 35/30

(52) U.S. Cl. 424/93.7

(57) ABSTRACT

A method for preparing a tissue culture insert that is used for constructing a transplantable graft of an engineered tissue equivalent comprising living main functional cells, stromal cells and tissue matrix on/in a biological supporting membrane for treatment of body tissue defects.

The present invention relates to a method of preparing a transplantable product for treatment of skin defects and claims the benefit of the Jun. 16, 2003 filing date of provisional application 60/478,427. The product contains supporting extraembryonic membranes and living cells.

### FIELD OF THE INVENTION

### The Prior Art

Terminology

Skin equivalent graft is prepared in a specific environment or chamber by combining keratinocytes with dermal fibroblasts and collagen supported by a supporting membrane.

Tissue culture insert provides an environment for the generation of polarized, high-density cultures, with adequate medium supply and the potential for establishing histotypic cell interactions.

An extraembryonic membrane consists of an amnion and chorion together. The chorion will be used refer to the outer layer of the extraembryonic membrane, the outer surface of which is in contact with the maternal deciduas during gestation. The terms of amnion and amniotic membranes will be reserved for the inner layer of the extraembryonic membrane as obtained when the amnion epithelium and its underlying layers are stripped from the chorion without using any elaborate techniques of separation, despite the fact that this does not result in an absolutely anatomical separation in histologic terms (R. N. Mathews et al., A Review Of The Role Of Amniotic Membranes In Surgical Practice. Obstet Gynaecol Annu. 1982, 11:31-58.).

Outer root sheath (ORS) cells are derived from the outer root sheath of human hair follicles. The cells represent a source of easily and repeatedly available keratinocytes, and thereby enables avoidance of dependence on surgery or suction blister material. These cells are especially suited for providing autologous keratinocytes (M. A. Pham et al. Reconstituted Epidermis: A Novel Model For The Study Of Drug Metabolism In Human Epidermis. J Invest Dermatol. 1990, 94(6):749-52.; A. Limat et al. Outer Root Sheath (ORS) Cells Organize Into Epidermoid Cyst-like Spheroids When Cultured Inside Matrigel: A Light-Microscopic And Immunohistological Comparison Between Human ORS Cells And Interfollicular Keratinocytes. Cell Tissue Res. 1994, 275(1):169-76.).

Epidermal cells are derived from the epidermis, which is the outermost layer of skin, and are composed of at least two cell types. The major cell type is the keratinocytes and a minor cell population is the melanocytes.

Dermal cells are derived from the dermis, which is the underline layer of epidermis of the skin. Dermal cells contain fibroblasts as the major cell type.

Transplantable sheets of living keratinous tissue is disclosed in U.S. Pat. No. 4,304,866.

U.S. Pat. No. 4,485,096 disclose tissue-equivalents and methods for preparation thereof.

Collagen compositions and methods for preparation of same are disclosed in U.S. Pat. No. 5,106,949.

U.S. Pat. No. 5,536,656 disclose the preparation of tissue equivalents by contraction of a collagen gel layered on a collagen gel.

U.S. Pat. No. 6,326,019 disclose grafts made from amnionic membrane; and methods of separating, preserving, and using such grafts in surgeries.

Anatomically and functionally, skin has two layers. The superficial epidermal layer provides a barrier against infection and moisture loss, whereas the epidermis consists of multiple layers of keratinocytes whose differentiation proceeds outward from a basal layer. The epidermis layer harbors three subpopulations of keratinocytes: stem cells, transient amplifying cells, and postmitotic differentiating cells (E. Christophers, Cellular Architecture Of The Stratum Corneum. J Invest Dermatol 1971, 56:165-169.; J. R. Bickenbach et al. Rate Of Loss Of Tritiated Thymidine Label In Basal Cells In Mouse Epithelial Tissues. Cell Tissue Kinet 1986, 19:325-333.). Stem cells and transient amplifying cells make up the proliferative pool of the epidermis. Stem cells are endowed with the potential to generate self-renewing tissues throughout a lifetime (L. G. Lajtha Stem Cell Concept. Differentiation. 1979, 14:23-34.; F. M. Watt. Stem Cell Fate And Patterning In Mammalian Epidermis. Curr Opin Genet Dev 2001, 11(4):410-7.; G. Cotsarelis et al. Epithelial Stem Cells In The Skin: Definition, Markers, Localization And Functions. Exp Dermatol, 1999, 8:80˜88.). The deeper dermal layer is responsible for the elasticity and mechanical integrity of the skin, and contains the bloods vessels that are responsible for the nutrition of the epidermal layer. Appendages, such as hair follicles or sweat glands, breach the epidermal and dermal layers. Cutaneous sensory nerves pass through the dermal tissue into the epidermal tissue. Regeneration of the epidermis relies on residues of epidermal cells that lie deep within dermal structures. During wound healing, in-growth of newly formed epidermis occurs from the edges of a wound and will be insufficient when the wound is more than a few cm across. In this event the wound would need a wound closure to assist in healing of the wound.

The wound closure requires a material to restore the epidermal barrier function and become incorporated into the wound during the healing and repair process. (R. G. Tompkins et al. Alternative Wound Coverings. In Herndon D, ed. Total Burn Care, 1^{st} ed. Philadelphia: W.B. Saunders Company Ltd, 1996:164-72.). The wound closure could be classified into two categories: temporary and permanent. Temporary wound closure can be achieved by using intact allograft and xenograft. Human amniotic membrane and different artificial biological membranes reviewed by (I. Jones et al. A Guide To Biological Skin Substitutes. British Journal of Plastic Surgery. 2002, 55:185-193.) have been used extensively as temporary wound closures in clinical treatment of skin injury. Temporary wound closures are most suited to superficial burns, where they create an improved environment for epidermal regeneration by providing a barrier against infection and water loss control, but are not useful for epidermal grafting. Using human amniotic membrane as a temporary wound cover has been documented since 1910 and reviewed by Mathews et al. below in 1982. Permanent wound closure results when the damaged area is loaded with epidermal cells. As the skin defect is repaired, the loaded epidermal cells provide the basis for new cell growth and wound skin reconstruction, prominently. This approach results in a skin substitute or skin equivalent graft. Skin substitutes currently available to the commercial market are artificial supporting membranes, having attached epidermal cells which are derived from allogenic skin; however, the allogenic graft often times does not have a permanent repairing function towards skin defects. Limited resources have resulted in the need to compare the clinical effectiveness and cost of such approaches. At this point, no real skin substitute for repairing skin defects and damage is available in the commercial market.

Human amniotic membrane is derived from the fetal membranes which consist of the inner amniotic membrane made of single layer of cuboidal amnion cells fixed to the collagen-rich basement membrane and outer mesenchyme tissue loosely attached to the chorion. It is composed of three layers: a single epithelial layer, thick basement membrane, and avascular stroma (R. N. Mathews et al. A Review Of The Role Of Amniotic Membranes In Surgical Practice. Obstet Gynaecol Annu. 1982, 11:31-58.). Human amniotic membrane has been shown to contain collagen types II and V It also contains collagen types IV and VII similar to corneal epithelial basement membrane as well as fibronectin and laminin. Additionally, it contains fibroblasts and other growth factors have been found in cryopreserved amniotic membranes (S. C. G Tseng, et al. Amniotic Membrane Transplantation for Ocular Surface Reconstruction. In: Ocular Surface Diseases: Medical and Surgical Management Ed. E. J. Holland et al. in press, 2001.). Amniotic membrane has been found to facilitate epithelialization, maintain a normal epithelial phenotype, and reduce inflammation, scarring, vascularisation, and the adhesion of tissues. Additionally, the membrane is believed to be nonimmunogenic. Antibodies or cell-mediated immune response to amniotic membrane have not been demonstrated, suggesting low antigenicity. Therefore, the use of systemic immunosuppressives in amniotic membrane transplantation is not required. Chorion has a surface which is composed of trophoblasts. The surface fused with maternal deciduas. There are pseudo-basement membrane and “collective tissue” layers underline of trophoblasts.

There is a need to devise a method for preparation of a cell culture insert using an extraembroynic membrane wherein allogenic/autologous epidermal cells and dermal cells can be seeded into the insert to construct a skin equivalent graft or substitute that is very similar with the human skin in the histological configuration and can be used for repairing skin defects permanently.

### SUMMARY OF THE INVENTION

One object of the invention is to provide a method which can produce a transplantable graft as a skin equivalent for repairing skin wounds and defects.

Another object of the invention is to provide a method which can produce a unique insert that contains a female slip slave vessel, male slip ring vessel and an extraembryonic biological supporting membrane for constructing a skin equivalent graft.

A further object of the invention is to provide a method which can produce a skin equivalent allograft made of extraembryonic membrane with allogeneic epidermal cells.

A still further object of the invention is to provide a method which can produce a skin equivalent autograft made of extraembryonic membrane with autologous epidermal cells or hair follicle cells.

A further object yet still of the invention is to provide a method which can produce an engineered transplantable graft based on the histological structures of human skin.

Another object of the invention is to provide a method which can produce a supporting membranes from an extraembryonic membrane as an elementary component for constructing skin equivalent grafts.

A further object of the invention is to provide a method which can prepare epidermal cells from skin as an elementary component of skin equivalent grafts.

A further object of the invention is to provide a method which can prepare dermal cells from skin as an elementary component of skin equivalent grafts.

Another object of the invention is to provide a method which can grow, select and amplify ORS cells from human hair follicles as an elementary component of skin equivalent grafts.

A further object of the invention is to provide a method which can prepare, store and transport samples of skin and hair follicles for preparing skin cells used in constructing skin equivalent grafts.

Another object of the invention is to provide a method which can prepare, store and transport an extraembroynic membrane for preparing the insert used in constructing skin equivalent grafts.

A still further object of the invention to provide a method which can prepare, store and transport skin equivalent grafts to users.

A further object yet still of the invention is to provide a method which can prepare a specific culture medium for stimulating proliferation of keratinocytes and thereby slow differentiation of keratinocytes on the insert.

Another object of the invention is to provide a method which can allow for detection and enrichment of immature keratinocytes important in the repair of skin defects.

A further object of the invention is to provide a method which can admit epidermal cell growth factor to promote epidermal cell growth on skin wounds.

Other objects of the invention will become apparent from the drawings and detailed description of the preferred embodiments of the invention.

### BRIEF DESCRIPTION OF THE DRAWINGS

FIG. 1 is a diagram illustrating the structure of supporting membrane, wherein A) demonstrates the anatomic structure of the extraembryonic membrane which can be split into the amnion and chorion from the separation layer; B) demonstrates the anatomic structure of the amnion which contains layers of Epithelium (1), Basement membrane (2), Compact layer (3), Fibroblast layer (4), and partial Spongy layer (5); and C demonstrates the anatomic structure of the chorion which contains the layers of partial Spongy layer (5), Cellular layer (6), Reticular layer (7), Pseudo-basement membrane (8), Trophoblast (9), and Maternal deciduas (10).

FIG. 2 is a diagram illustrating the preparation of supporting membrane; wherein A) and B) demonstrate the de-epithelial process of amnion; and C) and D) demonstrate the de-trophoblast process of chorion.

FIG. 3 is a diagram illustrating the preparation of an insert for constructing a skin equivalent; wherein A, B and C represent the basic elements of the insert including a “male” slip ring (A), “female” slip slave (C) and supporting membrane (B); D demonstrates small holes made for culture medium exchange; E and F show two chambers that are chamber A and B when the insert is assembled, and G represents the residual membrane after trimmed from the insert.

FIG. 4 is a diagram demonstrating the construction of a skin equivalent graft using the insert; wherein A) shows an assembled insert comprising a “female” slip slave, “male” slip ring and a sheet of supporting membrane; B), C) and D demonstrate seeding and culturing dermal cells (B) and epidermal cells (C) in the chamber A; D shows seeding and culturing dermal cells in chamber B; and E) demonstrates a constructed skin equivalent graft in the insert.

FIG. 5 illustrates a skin equivalent graft released from the insert; wherein A) shows the skin equivalent graft in the insert, B) shows the graft released when the insert is un-assembled; and C) demonstrates an intact graft under a phase contrast microscope at ×400. The skin equivalent graft contains epidermal layer (1), supporting membrane (2) and dermal cell layer (3).

FIG. 6 shows microscopic morphology of the supporting membranes and feeder cells; wherein A) shows epithelial part (EP) and de-epithelial part (BM) of human amnion under a microscope at ×150; B) shows trophoblast part (TB) and de-trophoblast part of human chorion; ×150; C) and D) shows morphology of feeder cells, 3T3 cells (C) and rat's embryonic fibroblasts (D) at ×225.

FIG. 7 shows part of a human hair follicle as source of the autologous epidermal cells in the graft. The human hair follicle has hair shaft (HS), outer root sheath (ORS) and inner root sheath (IRS), at ×100.

FIG. 8 is a diagram demonstrating amplification of human hair follicle cells through in vitro cell culture; wherein A), B) and C) show human ORS cells seeded (A), attached (B) and grown (C) on the feeder cells in the cell culture dish, at ×400; and D) shows one cell clone of keratinocytes from ORS cell, at ×225.

FIG. 9 is a diagram demonstrating amplification of epidermal cells prepared from skin through in vitro cell culture wherein A), B) and C) show epidermal cells seeded (A) at ×225, attached (13) and grown (C) at ×400 on the feeder cells in the cell culture dish; and D) shows pure epidermal cells after removing fibroblasts, at ×225.

FIG. 10 is a diagram demonstrating the construction of skin equivalent graft on the supporting membrane through cell culture in vitro; wherein A) shows bare amnion after de-epithelium without seeding cell, at ×225; B) demonstrates the amnion with dermal cells seeded, at ×225; C) shows the chorion with dermal cells seeded, at ×225; and D) shows the amnion with epidermal cells seeded, at ×225.

FIG. 11 shows skin equivalent graft; wherein A) shows epidermal cells on the supporting membrane after digestion of trypsin-EDTA, at ×225; B shows a repaired hole made on the amnion before seeding skin cells, at ×225; C) shows one skin equivalent graft (around 8 cm·sup·2) released from un-assembled insert; and D) demonstrates one skin equivalent graft (around 80 cm·sup·2).

FIG. 12 shows the characterization of epidermal cells detached from the skin equivalent graft; wherein A shows subculture of epidermal cells detached from skin equivalent graft, at ×225; B) shows cytokeratin 19 positive epidermal cells detached from skin equivalent graft, at ×945; C shows integrin beta-1 positive epidermal cells detached from the skin equivalent graft, at ×945; and D) shows normal kerotype of the epidermal cells detached from the skin equivalent graft.

### DESCRIPTION OF PREFERRED EMBODIMENTS OF THE INVENTION

In accordance with the present invention, a skin equivalent graft comprises three basic elements which consists of the epidermal cell sheet containing: 1. keratinocytes, 2. melanocytes; and a dermal cell sheet containing 3. fibroblasts and a supporting matrix membrane derived from an extraembroynic membrane. Keratinocytes can be derived from both allogeneic and autologous skin or hair follicles.

Keratinocytes in the graft comprises three basic cell types of stem cells, transient amplifying cells, and postmitotic differentiating cells. Stem cells and transient amplifying cells make up the proliferative pool of the epidermis. Stem cells are endowed with the potential to generate self-renewing tissues throughout its lifetime. A stem cell population can be detected by using the method in the invention as an indicator of the quality of the graft.

A new cell culture insert which is able to be assembled and un-assembled has been specifically designed for producing the graft. The insert can be used to mount a supporting membrane such as extraembroynic membrane. Two surfaces of the supporting membrane can be exposed into two separate chambers. The cells can be seeded and cultured on either surface of the supporting membrane.

The method of the present invention has been implemented using the insert to produce a graft which is a skin substitute, that is engineered to have a similar configuration to human skin. The different layers of the graft are formed by seeding different cells into different chambers of the insert at different cell culture periods.

The new cell culture insert of the invention is specifically designed to produce a graft. The insert consists of three parts which include a set of freely assembled and un-assembled parts and a sheet of supporting membrane. An intact insert has two chambers separated by the supporting membrane. Two surface sides of the supporting membrane are directly exposed into two separated chambers. The cells are then seeded and cultured on any surface side of the supporting membrane through the insert chambers.

I. Brief Description of Processes for Preparing the Transplantable Graft Base on the Drawings.

Preparing Biological Supporting Membrane

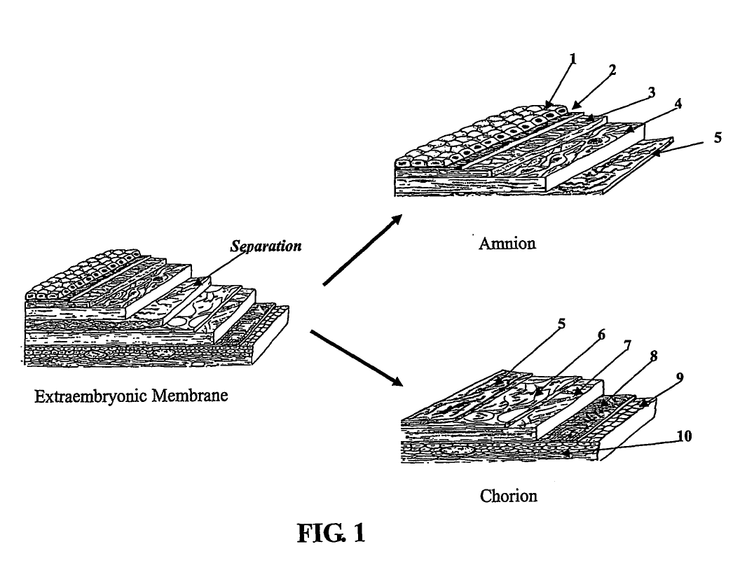

FIG. 1 is a diagram illustrating the structure of supporting membrane, wherein A) demonstrates the anatomic structure of the extraembryonic membrane which can be split into the amnion and chorion from the separation layer; B) demonstrates the anatomic structure of the amnion which contains layers of Epithelium (1), Basement membrane (2), Compact layer (3), Fibroblast layer (4), and partial Spongy layer (5); and C demonstrates the anatomic structure of the chorion which contains the layers of partial Spongy layer (5), Cellular layer (6), Reticular layer (7), Pseudo-basement membrane (8), Trophoblast (9), and Maternal deciduas (10).

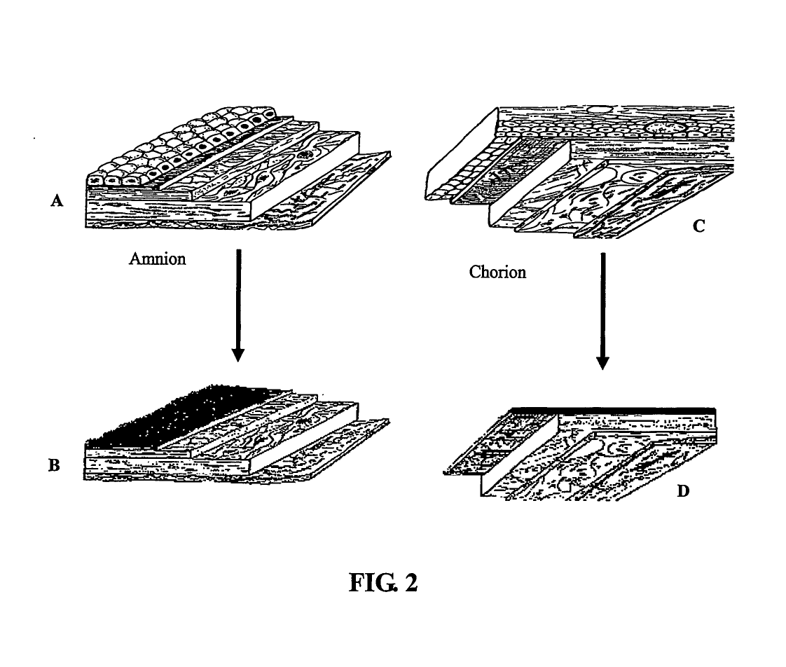

FIG. 2 is a diagram illustrating the preparation of supporting membrane; wherein A) and B) demonstrate the de-epithelial process of amnion; and C) and D) demonstrate the de-trophoblast process of chorion.

Preparing a Specific Tissue Culture Insert and Constructing the Graft

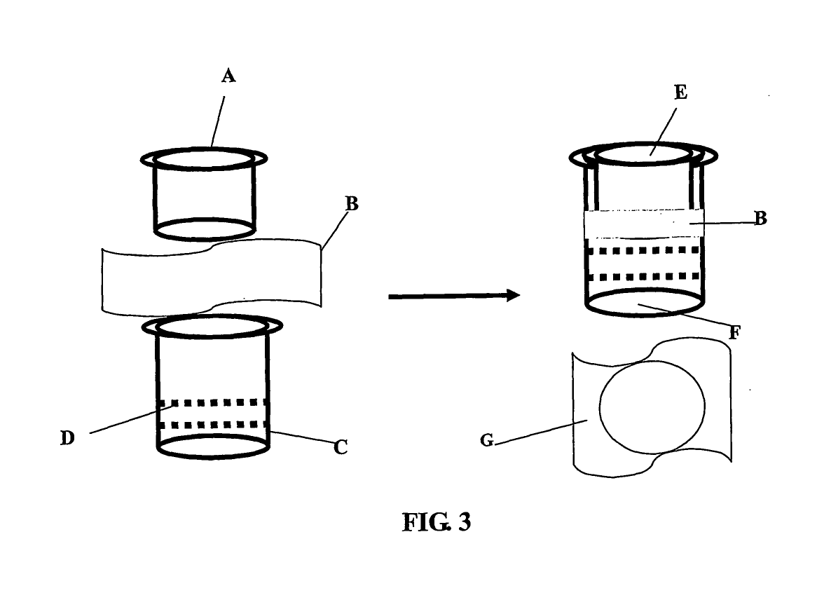

FIG. 3 is a diagram illustrating the preparation of an insert for constructing a skin equivalent; wherein A, B and C represent the basic elements of the insert including a “male” slip ring (A), “female” slip slave (C) and supporting membrane (B); D demonstrates small holes made for culture medium exchange; E and F show two chambers that are chamber A and B when the insert is assembled, and G represents the residual membrane after trimmed from the insert.

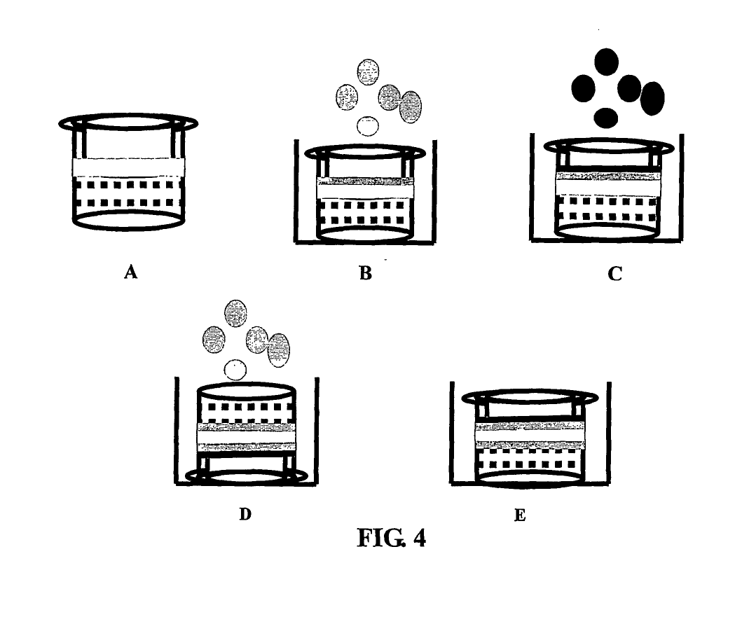

FIG. 4 is a diagram demonstrating the construction of a skin equivalent graft using the insert; wherein A) shows an assembled insert comprising a “female” slip slave, “male” slip ring and a sheet of supporting membrane; B), C) and D demonstrate seeding and culturing dermal cells (B) and epidermal cells (C) in the chamber A; D shows seeding and culturing dermal cells in chamber B; and E) demonstrates a constructed skin equivalent graft in the insert.

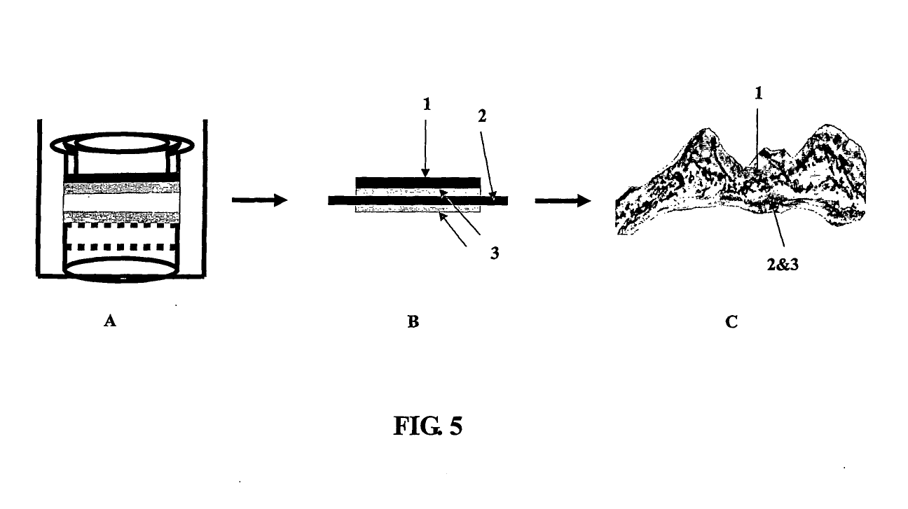

FIG. 5 illustrates a skin equivalent graft released from the insert; wherein A) shows the skin equivalent graft in the insert, B) shows the graft released when the insert is un-assembled; and C) demonstrates an intact graft under a phase contrast microscope at ×400. The skin equivalent graft contains epidermal layer (1), supporting membrane (2) and dermal cell layer (3).

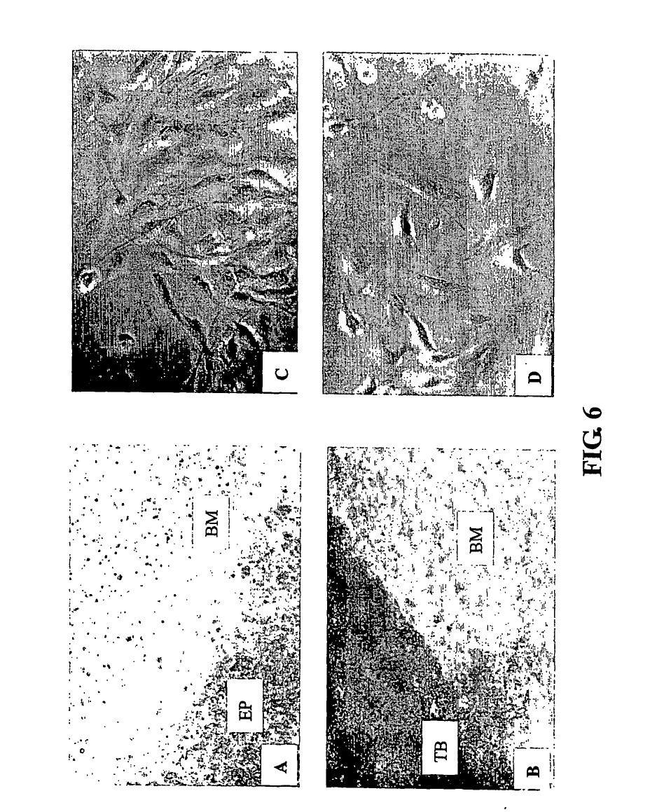

Preparing supporting membrane and feeder cells FIG. 6 shows microscopic morphology of the supporting membranes and feeder cells; wherein A) shows epithelial part (EP) and de-epithelial part (BM) of human amnion under a microscope at ×150; B) shows trophoblast part (TB) and de-trophoblast part of human chorion; ×150; C) and D) shows morphology of feeder cells, 3T3 cells (C) and rat's embryonic fibroblasts (D) at ×225.

Preparing Seed Cells and their Quality Control

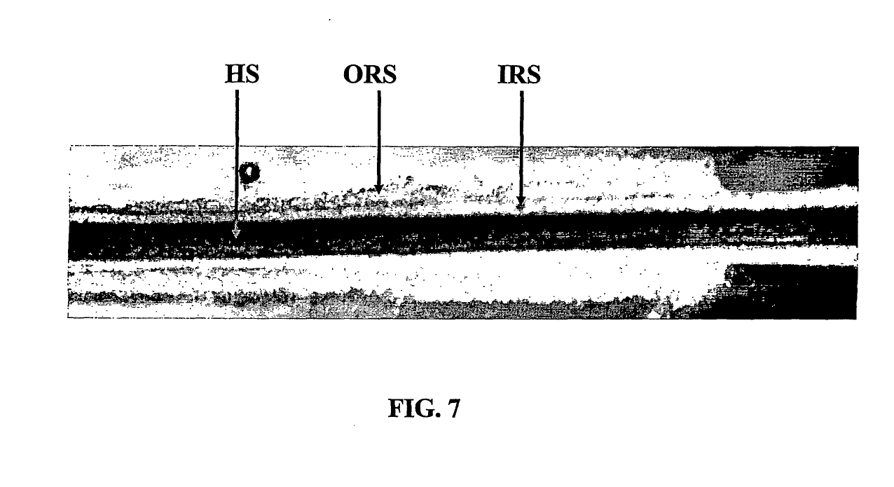

FIG. 7 shows part of a human hair follicle as source of the autologous epidermal cells in the graft. The human hair follicle has hair shaft (HS), outer root sheath (ORS) and inner root sheath (IRS), at ×100.

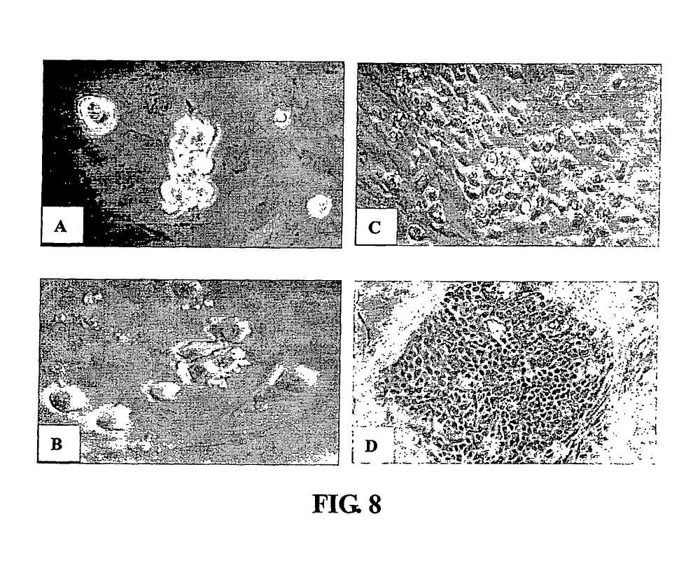

FIG. 8 is a diagram demonstrating amplification of human hair follicle cells through in vitro cell culture; wherein A), B) and C) show human ORS cells seeded (A), attached (B) and grown (C) on the feeder cells in the cell culture dish, at ×400; and D) shows one cell clone of keratinocytes from ORS cell, at ×225.

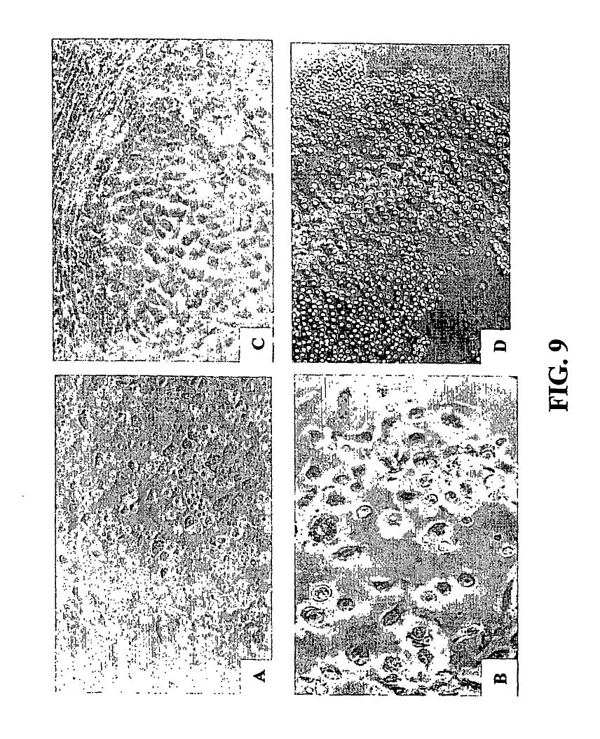

FIG. 9 is a diagram demonstrating amplification of epidermal cells through in vitro cell culture wherein A), B) and C) show epidermal cells seeded (A) at ×225, attached (B) and grown (C) at ×400 on the feeder cells in the cell culture dish; and D) shows pure epidermal cells after removing fibroblasts, at ×225.

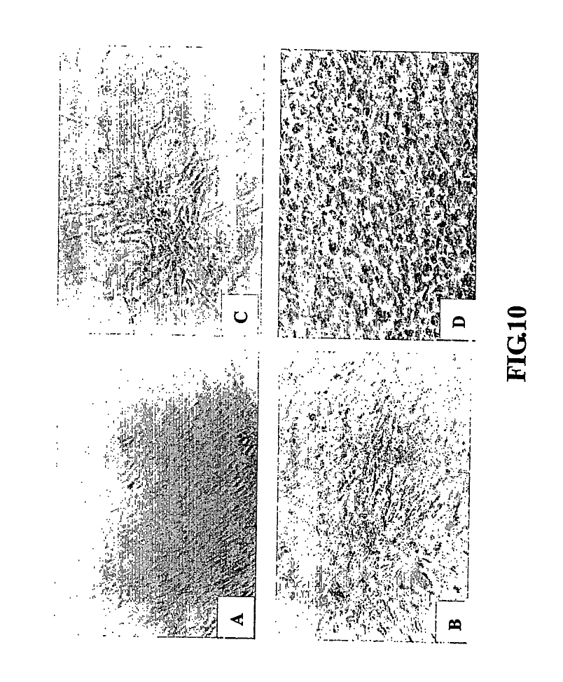

FIG. 10 is a diagram demonstrating the construction of skin equivalent graft on the supporting membrane through cell culture in vitro; wherein A) shows bare amnion after de-epithelium without seeding cell, at ×225; B) demonstrates the amnion with dermal cells seeded, at ×225; C) shows the chorion with dermal cells seeded, at ×225; and D) shows the amnion with epidermal cells seeded, at ×225.

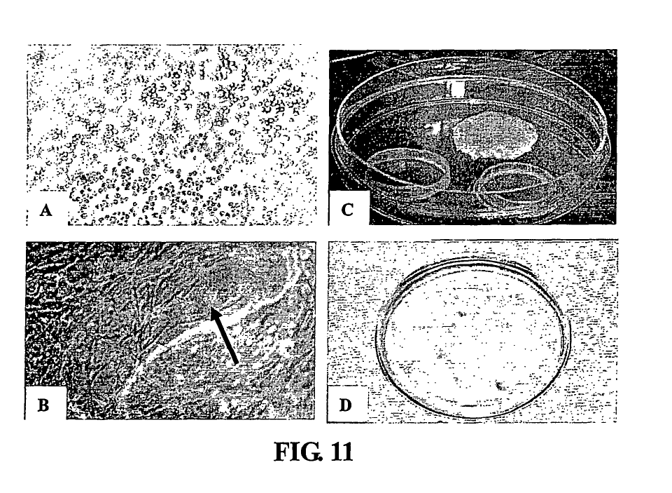

FIG. 11 shows skin equivalent graft; wherein A) shows epidermal cells on the supporting membrane after digestion of trypsin-EDTA, at ×225; B shows a repaired hole made on the amnion before seeding skin cells, at ×225; C) shows one skin equivalent graft (around 8 cm²) released from un-assembled insert; and D) demonstrates one skin equivalent graft (around 80 cm²).

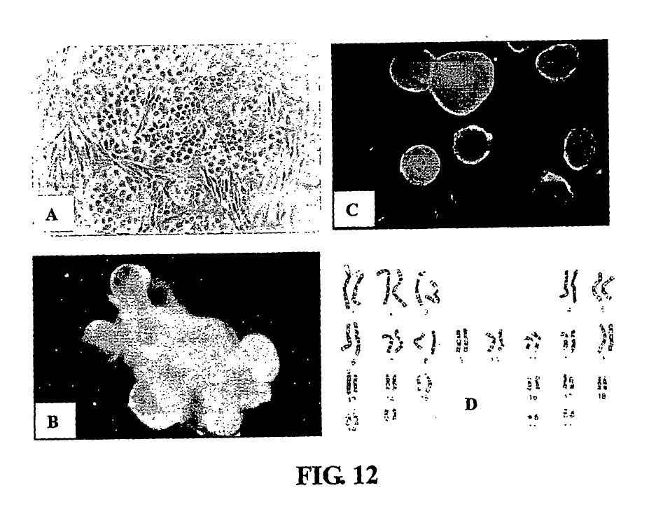

FIG. 12 shows the characterization of epidermal cells detached from the skin equivalent graft; wherein A shows subculture of epidermal cells detached from skin equivalent graft, at ×225; B) shows cytokeratin 19 positive epidermal cells detached from skin equivalent graft, at ×945; C shows integrin beta-1 positive epidermal cells detached from the skin equivalent graft, at ×945; and D) shows normal kerotype of the epidermal cells detached from the skin equivalent graft.

II. Preparation of an Insert for Construction of the Graft

A design for producing the specific insert for preparation of the graft is based on the design of cell culture dishes available to the current cell culture market, and a “tissue” engineering configuration of the graft prepared in the present invention. Designs of cell culture dishes are commercially available and as classified into several categories based on the size of the dish diameter: 35 mm, 60 mm, 100 mm and 150 mm. The insert can also be manufactured to fit in culture plates with 6-wells, 12-wells, 24-wells and 48-wells.

The insert is specifically made for producing a transplantable graft which contains at least one sheet of living cells. The insert contains three parts (see FIG. 3) which are a “female” slip slave C, a “male” slip ring A, and a sheet of supporting membrane B. Based on the size of cell culture dish or plate-well, the insert can be made in different sizes. The “female” slip slave is manufactured from 5 mm to 200 mm in height and from 5 mm to 150 mm in diameter. Small holes on the wall or “V” shaped cuts in the bottom of the slave can be made for creating a pathway for cell culture medium to flow from outside into the inside of the chamber when the insert is used in a cell culture dish along with a culture medium.

The height of “male” slip ring is a half the height of the “female” slip slave. The “male” slip ring has a collar width 1 to 2 mm thick at one end, and can be inserted into the inside of “female” slip slave. On the surface of “male” slip ring, there are longitudinal spacing bridges. The bridges tightly contact the inside wall of “female” slip slave when both parts are assembled. There are small gaps between two spacing bridges and between the inside surface of the “female” slip slave and the outside surface of the “male” slip ring.

One piece of the supporting membrane covers the top of “female” slip slave. Next, the “male” slip ring can be used to insert the membrane into the “female” slip slave. Any remaining pieces of the supporting membrane is cut by an ophthalmologic scissor. The edge of the supporting membrane is stretched and clipped between the “male” and the “female” pieces of the assembly. The supporting membrane installed in the insert creates two chambers; an upper called chamber A formed using the upper surface of the membrane as the bottom and the inside of “male” slip ring as the wall of the chamber A. and a lower chamber B, which is a reverse surface of the supporting membrane that is a top of chamber B and the inside of the “female” slip slave serves as the wall of the chamber B.

The intact insert assembly using the three parts mentioned above creates two cell culture chambers (chamber A and B) and two surfaces of supporting membrane are exposed to the chamber A and B, separately.

III. Preparation of the Supporting Membrane—Extraembryonic Membrane:

Sampling

In order to avoid any potential blood-transmittable diseases, seronegative healthy mothers in a prescreening of HIV-1, HIV-2, HTLV-1, hepatitis B and C viruses and syphilis, are chosen as donors of membrane. Tissue taken at the time of cesarean section is ideal. The extraembryonic membrane is removed by trimming them from the placenta at the time of delivery, either vaginal or by cesarean section. Maternal blood, meconium, and other contaminants are removed by washing in sterile saline until grossly clean and then, transferred in an ice bucket to the laboratory as soon as possible.

Preservation and Transportation of the Membrane

Extraembryonic membrane is taken from the saline and saline is drained away from the membrane. Three hundred ml of 85% alcohol are filtered and prepared for each extraembryonic membrane. The membrane is transferred into the alcohol for fixation of 24 hours to 48 hours. After fixation, the membrane is removed from alcohol into a 300 ml sterilized saline for ten-minutes of washing. The wash is repeated at least three times, and the operation is carried out under sterilized criteria of cell culture in the cell culture hood. Subsequently, the membrane is trimmed into the size of requirements and soaked in a cryopreservation solution in sterilized polystyrene tubes. The cryopreservation solution contains 50 percent of glycerol (Sigma) and 50 percent of DMEM culture medium (ATCC). An alternative cryopreservation solution also used in the invention contains 20 (5-50) percent of DMSO (Sigma) and 80 percent of DMEM to ensure more than one-year of preservation of the membrane. The membrane in the cryopreservation solution can be stored at 4° C. for one month in a refrigerator, or in a freezer at −20° C. for more than one year.

For fresh use, the trimmed extraembryonic membrane is only soaked into DMEM supplemented with 250 U/ml penicillin, 250·mu·g/ml streptomycin, 100·mu·g/ml kanamycin or 50·mu·g/ml gentamycin, and 2.5·mu·g/ml amphotericin and stored at 4° C. within 48 hours.

Preparation of Amnion and Chorion

A tube with the freezing membrane is placed into a refrigerator at 4° C. overnight. The membrane is then transferred into a DMEM culture medium for at least three washing in a sterilized culture dish in a cell culture hood for ready separation of the amnion and chorion.

Two sets of forceps and two pairs of ophthalmologic scissors are prepared by autoclaving sterilization. One flaming light is prepared in the cell culture hood and used for quick sterilization in case of contamination of any instruments during the operation of separation of the membranes.

With two sets of forceps, the extraembryonic membrane is easily separated from a sponge layer between amnion and chorion (See FIG. 1; separation) by blunt dissections, while immersed in the DMEM cell culture medium. The separated amnion, as a sheet, then is mounted in the insert described above. The epithelial surface of the amnion is kept facing up towards chamber A and the connective tissue side is kept facing down towards chamber B. The separated chorion, as a sheet, is further trimmed to remove the layer of maternal decidue and mounted in another insert. The trophoblast surface is kept facing toward chamber A and “connective tissue” side is kept facing toward chamber B of the insert.

Preparation of the Supporting Membranes from the Amnion

As described above, amnion is selected as a supporting membrane and assembled into the insert. The epithelial surface of the amnion is made to face the chamber A and the reverse connective tissue surface is made to face the chamber B. When the insert is completely assembled, the epithelial layer of the amnion is removed by digestion with 0.25% trypsin with EDTA (ethylenediaminetetraacetate) (Sigma) at 37° C. for 10 to 30 minutes, and subsequently scraping it off with a rubber cell scraper. The cellular debris detached from the amnion is discarded and washed with DMBM until clean. The operation of removing epithelium avoids causing serious damage of the basement membrane which is underline of the epithelial layer of amnion. Completion of the de-epithelium process makes it ready for seeding the cells or further enhanced treatment (see FIGS. 2 A&B).

Preparation of the Supporting Membranes from the Chorion

The separated chorion can also be a supporting membrane which is assembled into the insert. The method for preparation of the chorion insert is the same as the preparation of the amnion insert. The trophoblast surface is oriented in the chamber A and “connective tissue” side is made to face toward chamber B. Digestion using 0.25% trypsin with EDTA is carried out in chamber A at 37° C. for 10 to 30 minutes. The trophoblast layer is then removed by scraping with a rubber cell scraper. A pseudo-basement membrane is then exposed to chamber A and the “connective tissue” side is exposed in chamber B. The membrane is then ready for seeding and culturing skin cells (see FIGS. 2 C&D).

When the epithelial layer or trophoblast layer is removed by trypsin digestion and mechanical scratching, the basement membrane is easily damaged and damage of basement membrane influences the cell adherence and growth on the supporting membrane. Accordingly, the membrane needs to be coated using an enhancing treatment.

IV. Enhancing Treatment of the Supporting Membrane

Preparation of Crude Collagen Solution

Collagen is derived from rat tail tendon and calf tendon collagen. Other sources of collagen including human fetal skin have been employed; however, and still other sources would be suitable in the context of the invention. Solutions of collagen are prepared and maintained under slightly acidic conditions. The collagen solutions are prepared as follows. Frozen rat tails from 450 gm rats are thawed in 70% EtOH for 20 minutes. The tendon bundles are excised in 70% EtOH in a laminar flow hood. Individual tendons are pulled out of the tendon sheath, minced and placed in 0.1M acetic acid using 250 ml per tail. This solution is left standing for 48 hours at 4° C. at which point the minced tendons have swelled to occupy the total volume. This viscous solution is centrifuged at 15 k rpm in a Sorvall centrifuge for half an hour. The supernatant is drawn off and mixed with 0.1 M NaOH in a 4:1 ratio to neutralize the acetic acid, upon which collagen precipitated. This solution is centrifuged at 3000 rpm for 15 minutes in a centrifuge. The supernatant is discarded and an equal volume of fresh 0.01 M acetic acid is introduced to resolubilize the collagen. This solution is stored at 4° C. as a coating collagen solution. The quantity of protein in the solution is around 3 mg/ml.

Preparation of Cell Growth Supplementary Solution

DMEM cell culture medium is supplemented with a final concentration of 10% fetal calf serum, 0.1 mM CaCl₂, 4 mM L-glutamine, 100 U/mL penicillin, 100·umg/mL streptomycine sulfate, 0.25·mu·g/mL amphotericin B, 0.4·mu·g/mL hydrocortisone, 10⁻¹⁰M cholera enterotoxin, 5·mu·g/mL transferrin, 2 [times]×10⁻¹¹M liothyronine, 1.8×10⁻⁴ M adienine, 5·mu·g/mL insulin, and 10 ng/mL epithelial growth factor. The solution is concentrated three-fold, aliquoated and kept at −80° C. in a freezer in present invention.

Basement Membrane Element Enhancing Solution

One solution for enhancing basement membrane elements contains 0.4 mg/ml collagen IV (from Life Technology), 1.0 mg/ml fibronactin (from Life Technology) and 1.0 mg/ml laminin (from Life Technology).

Enhancing Solution

For coating each 10 cm area of the supporting membrane there is needed 100·mu·L (from 20 to 150·mu·L) of enhancing solution which contains 40·mu·L of collagen solution, 30·mu·L of cell growth supplementary solution and 30·mu·L of basement membrane element enhancing solution that is made fresh.

Coating Membrane

In order to enhance the cell adherence and growth of cells on the membrane, the membrane is modified as follows on both membrane surfaces. The assembled insert with membrane is washed twice with DMEM. Following washing the insert is transferred into an empty culture dish without culture medium. The dish is placed in an incubator at 37° C. for at least two hours to allow the membrane to dry completely.

After the membrane is completely dried, the enhancing solution (0.2 ml/10 cm² surface area of chamber) is added to side A of the chamber, containing the basement membrane surface. Following complete wetting of the membrane surface, the remaining enhancing solution is removed from the membrane surface by aspiration. Next, the insert is placed upside down to expose the other chamber or chamber B upward. The same procedure is performed on side B as was performed on side A of the chamber. The coated supporting membrane is air dried in a biological safety cabinet for one to two hours. The insert is now ready for seeding and culturing the cells.

V. Sampling for Preparation of Seeding Cells

Sampling

Sampling of Skin

Skin samples were donated from allogeneic and autologous sources. The skin specimens can be collected from surgery. Neonatal as well as juvenile foreskin samples are commonly used. Larger samples can be obtained from a postmortem (up to 48 hours) of abdominal skin. The skin samples can also be made available from aborted fetuses. The density of the hypodermis varies with the biopsy site. For foreskins, the hypodermis is particularly loose and therefore easily dissectible, whereas skin taken from the back has an extremely dense hypodermis, which proves difficult to remove. In the latter case, as much extraneous connective tissue as possible is removed. The skin is often contaminated with bacteria or yeast, or fungus. Submerging the skin sample in alcohol before processing should kill most forms of contamination. However, pockets of bacteria which have become trapped in sweat or sebaceous pores may be present. Foreskins are particularly prone to blocked pores. Fortunately, once the skin is stretched upside down across the Petri dish, the presence of blocked pores is usually obvious. The affected areas of skin should be carefully dissected out and discarded, taking particular care not to cut onto the blocked pore.

To prevent infection of cultures, the skin samples should be rinsed 5 to 10 times in DMEM culture medium with antibiotics: 250 U/ml penicillin, 250·mu·g/ml streptomycin, 100·mu·g/ml kanamycin or 50·mu·g/ml gentamycin, and 2.5·mu·g/ml amphotericin. The skin samples are then transported in the transport medium (described in following section) to the laboratory.

Sampling of Hair Follicles

Keratinocytes can be sourced from the cell culture of outer root sheath (ORS) of hair follicles. The outer root sheath of hair follicles is a multilayered tissue made up predominantly by undifferentiated keratinocytes, and can contribute to the regeneration of the epidermis.

Scalp hair follicles are preferred to be isolated from the occipital region, but can be also isolated from the beard, leg, and genital region. The region selected is sterilized by spraying 75% alcohol and waiting for 5 minutes before starting sampling. The hairs to be plucked are exposed by pulling up the adjacent hair. A few numbers of hairs (maximally 8 to 10) are gripped with gross sterile forceps as close as possible to the skin surface. The hairs are pulled out by a jerky movement made perpendicular to the skin surface. The follicle material is then directly collected into a 60-mm bacteriological dish containing 5 ml of a rinsing medium, by cutting with fine sterile scissors. The remaining distal keratinized hair shaft is discarded. At least one follicle has to be prepared per final milliliter of culture medium. The follicles in the anagen phase are selected under a dissecting stereomicroscope and transferred into a new 60-mm bacteriological dish containing 5 mL of rinsing medium. The bulbar part can be kept and the distal fifth of the follicular length corresponding to the infundibular part using miniscalpels, which ensures that the only living cell population in the remaining follicle is constituted by outer root sheath cells. The prepared follicles are rinsed four times by consecutive transfers in 60-mm bacteriological dishes containing 5 mL of rinsing medium. The rinsing medium contains PBS (Phosphate-Buffered Saline) with antibiotics: 250 U/ml penicillin, 250·mu·g/ml streptomycin, 100·mu·g/ml kanamycin or 50·mu·g/ml gentamycin, and 2.5·mu·g/ml amphotericin.

Transportation of Human Hair Follicles

The prepared hair follicles described above are put in a freezing medium which contain DMEM with 30% FBS, antibiotics and 5% DMSO. The samples are gradually frozen in dry ice at −80° C. and kept at −80° C. during transportation.

VI. Compositions of Cell Culture Media

Transportation Medium

For transportation of skin or hair follicles, DMEM is supplemented with 10% FCS, 100 U/mL penicillin, 100·mu·g/mL streptomycin, 0.25·mu·g/mL amphotericin B, 50·mu·g/mL gentamicin. The medium is stored at 4° C. and medium supplements are stored as concentrated stocks at −20° C.

Epidermal Cell Primary Culture Medium (EPM)

Epidermal Cell Primary Culture Medium is made up of a Minimum Essential Medium Eagle (from Sigma), and supplemented with 10% fetal calf serum, 0.1 mM CaCl.₂, 4 mM L-glutamine, 100 U/mL penicillin, 100·umg/mL streptomycine sulfate, 0.25·mu·g/mL amphotericin B, 0.4·mu·g/mL hydrocortisone, 10⁻¹⁰M cholera enterotoxin, 5·mu·g/mL transferrin, 2×10⁻¹¹ M liothyronine, 1.8×10⁴M adinine, 5·mu·g/mL insulin, and 10 ng/mL epithelial growth factor. This medium is used fresh if possible, but has a shelf life of approximately 1 week. The supplements are stored as concentrated stock at −20° C.

Keratinocyte Growth Medium (KGM)

Keratinocyte Growth Medium is made up of a 1:3 (v/v) mixture of Ham's F12 and DMEM media (from Sigma), and supplemented with 10% fetal calf serum, 4 mM L-glutamine, 100 U/mL penicillin, 100·umg/mL streptomycine sulfate, 0.25·mu·g/mL amphotericin B, 0.4·mu·g/mL hydrocortisone, 10⁻¹⁰ M cholera enterotoxin, 5·mu·g/mL transferrin, 2×10⁻¹¹ M liothyronine, 1.8×10⁻⁴ M adinine, 5·mu·g/mL insulin, and 10 ng/mL epithelial growth factor. This medium is used fresh if possible, but has a shelf life of approximately 1 week. The supplements are stored as concentrated stock at −20° C.

ORS Cell Primary Culture Medium (ORSM)

ORS Cell Primary Culture Medium is made up of a 25:75 (v/v) mixture of Ham's F12 and DMEM media (from Sigma), and supplemented with 10% fetal calf serum, 4 mM L-glutamine, 100 U/mL penicillin, 100·umg/mL streptomycine sulfate, 0.25·mu·g/mL amphotericin B, 0.4·mu·g/mL hydrocortisone, 10⁻¹⁰M cholera enterotoxin, 5·mu·g/mL transferrin, 2×10⁻¹¹ M liothyronine, 1.8×10⁻⁴ M adinine, 5·mu·g/mL insulin, and 10 ng/mL epithelial growth factor. This medium is used fresh if possible, but has a shelf life of approximately 1 week. The supplements are stored as concentrated stock at −20° C.

Feeder Cell Culture Medium

Feeder cell culture medium is made up DMEM and supplemented with 10% fetal calf serum, 4 mM L-glutamine, 100 U/mL penicillin, 100·umg/mL streptomycine sulfate, 0.25·mu·g/mL amphotericin B.

VII. Culture and Amplification of Seeding Cells

Preparation of Feeder Cell Layer

The technology for preparation in vitro of keratinocytes has been described in detail by several prior authors (Y. Barrandon et al. Cell Size As A Determinant Of The Clone-Forming Ability Of Human Keratinocytes. Proc Natl Acad Sci USA. 1985, 82(16):5390-4.; Y Barrandon et al. Three Clonal Types Of Keratinocyte With Different Capacities For Multiplication. Proc Natl Acad Sci USA. 1987, 84(8):2302-6.; M. B. Mathor et al. Clonal Analysis Of Stably Transduced Human Epidermal Stem Cells In Culture. Proc Natl Acad Sci USA. 1996, 93(19):10371-6.; A. Rochat et al. Location Of Stem Cells Of Human Hair Follicles By Clonal Analysis. Cell. 1994, 76(6):1063-73.; F. M. Watt et al. Epidermal Stem Cells: Markers, Patterning And The Control Of Stem Cell Fate. Philos Trans R Soc Lond B Biol Sci 1998, 353(1370):831-7)

Two kinds of feeder cells are used in the invention. They are human fibroblasts and 3T3 cell line (from ATCC). 3T3 cells are adherent cells and grown in the feeder cell culture medium at 37° C. to near confluence (around 70%) and then, fresh culture medium is changed with 1 to 10·mu·g/mL of mitomycin-C and incubated for a further 5 to 12 hours. The cells treated by mitomycin-C can be harvested immediately by trypsinization in the usual manner and seeded in a culture dish coated by collagen at approximately 2.5×10⁴ cells/cm². The dish can be used for keratinocyte cell culture after adherence of the 3T3 cells.

Human fibroblasts are prepared from surgical samples or from abortion fetal samples. The fibroblasts are generated from digestion of connective tissues and cultured primarily in the feeder cell culture medium for passing multiple generations. The cells are then used for feeding skin cells. Preferably, the invention uses human dermal fibroblasts as feeding cells. Culture of human dermal fibroblasts are propagated in EMEM supplemented with 10% FCS with a weekly split ratio of 1:2, best in 100-mm culture dishes. Subcultivation is done with 0.05% trypsin-0.02% EDTA in PBS without calcium and magnesium at 37° C. for approximately 5 minutes. Trypsin is inactivated by the addition of 3.5 mL DMEM supplemented with 10% FCS. To prepare feeder layers, continent cultures are subcultured in a 1:4 ratio instead of the usual 1:2 ratio, and incubated overnight at 37° C. in the CO₂ incubator. The next day these Fibroblasts are rendered postmitotic by treatment for 5 hours at 37° C. in the CO₂ incubator with 8·mu·g/mL mitomycine-C in DMEM supplemented with 10% FCS. After 5 hours of incubation, the cells are rinsed at least four times with PBS containing calcium and magnesium, followed by one rinse with PBS without calcium and magnesium. The fibroblasts are detached by treatment with 0.05% trypsin-0.02% EDTA in PBS without calcium and magnesium at 37° C. for approximately 3 minutes. Detachment of the cells can be speeded up by carefully agitating the culture dish. The trypsin is blocked by the addition of 4.5 mL of DMEM containing 10% FCS/100-mm tube, after which a cell count of the suspension is performed. The suspension is diluted in DMEM containing 10% FCS so as to obtain a suspension of 2×10⁴ cells/mL. The suspension is then distributed in the selected culture dishes, usually in 35-mm culture dishes. The now ready-to-use feeder layers can be stored at 37° C. in the CO₂ incubator for at least 20-30 days with a weekly medium change until use. For long-term storage, feeder cells can be kept frozen in liquid nitrogen. For this purpose, 1×10⁶ mitomycine-C treated fibroblasts are suspend in 1 mL of DMEM containing 10% FCS and 10% dimethyl sulfoxide (from Sigma), transferred to a 1.8-mL cryotube, left inside a styropor box at −80° C. for 24 hours, and finally transferred into the liquid nitrogen tank. A reproducible plating efficiency of 50% is obtained when recovering the frozen cells from the cryogenic storage. For preparation of feeder layers, the fibroblasts are suspended at a density of 4×10⁴ cells/mL medium.

Preparation of Coated Cell Culture Dishes

Fibrenestin, luminin and collagens can be used for preparation of coated cell culture dishes. The collagen IV is preferred in present invention.

Preparation of the Seeding Cells from Intact Skin

Before processing, the skin is removed from the transport media, washed in PBS, submerged briefly in 70% alcohol three times, and shake-dried in a tissue-culture hood. The skin is placed into a shallow sterile container (a 10-cm Petri dish is perfect for small skin samples). Forceps and iris scissors are used to trim away the hypodermis and the skin remains a relatively dense dermis. The skin is flattened and the epidermis is placed onto the surface of the Petri dish, whereupon is used to a sterile scalpel cut the skin into 2 to 3 mm pieces. The sample is placed into a universal container containing at least a covering amount of 0.25% trypsin-EDTA (from Sigma), and incubated overnight at 4° C. for 12 to 48 hours. After incubation, the sample is removed from the trypsin solution by filtration using a sieve. A DMEM with 10% FCS is added to inactivate the trypsin. The cells of the sample were dispersed by pipeting. The single-cell suspension after filtration by a sieve with 100·mu·m meshes was prepared for cell counting using a hemocytometer under a microscope. A cell pellet was formed by centrifugation at approximately 300 g for 5 minutes, and resuspended in the freezing medium based on the cell density of 2.0-5.0×10⁶ cells per ml. The cells were stored at −80° C. for seeding on the supporting membrane.

Preparation of Epidermal Cells and Dermal Cells from the Skin

The procedure for preparing epidermal and dermal cells separately is similar to the procedure for preparation of the cells from intact skin. Before cold digestion of trypsin-EDTA, the skin is flattened to make dermis onto the surface of the Petri dish and a sterile scalpel is used to cut the skin into long thin strips. After cold digestion incubation, the strips of skin are removed from the digestion medium and excess media is dabbed off on the inside of the lid of a 10-cm Petri dish and the relatively media-free strips are placed into the Petri dish. When the first detachment of epidermis is visible at the cut edges of skin samples, the pieces are placed (dermis-side down) in a 10-cm complete culture medium including serum. With two fine curved forceps the epidermis is gently peeled of and pooled in a 50-ml centrifuge tube containing 20 ml of complete culture medium. Viable keratinocytes are detached from the epidermal part by vigorous pipetting and sieving through a nylon gauze (100·mu·m mesh). The remaining dermal part is gently scraped with curved forceps on its epidermal (upper surface) to remove loosely attached basal cells. The cells detached from the basal membrane, and keratinocytes from the epidermis were mixed and passed through a nylon gauze (100·mu·m mesh), washed twice in culture medium by centrifugation at 100 g for 10 minutes, and counted for total and viable (trypan blue excluding) cells. Scraping of the dermal surface yields higher cell numbers. Moreover, when trypsinization is performed at 37° C., scraping of the upper surface of the dermis does not substantially increase cell yield, since splitting occurs mostly at the basal lamina. The cell pellet was formed by centrifugation at approximately 300 g for 5 minutes, and resuspended in the freezing medium based on the cell density of 2.0-5.0×10⁶ cells per ml. The cells were stored at −80° C. for seeding on the supporting membrane.

Dermal layer split from epidermal layer may further be digested in the trypsin-EDTA solution at 37° C. for 10 to 15 minutes. The dermis is then transferred into completed culture medium and the cells dispersed by a Pasteur pipet. The isolated cells from the dermal were filtered through a nylon gauze (100·mu·m mesh), washed twice in culture medium by centrifugation at 100 g for 10 minutes, and counted for total and viable (trypan blue excluding) cells. A cell pellet was formed by centrifugation at approximately 300 g for 5 minutes, and resuspended in the freezing medium based on the cell density of 2.0-5.0×10⁶ cells per ml. The cells were stored at −80° C. for construction of the graft.

Preparation of the Seeding Cells from Hair Follicle:

Isolation of the ORS Cells from the Follicles

The follicles are deposited onto an empty 35-mm bacteriological dish in such a way that they are in close vicinity, though separated from each other. This guarantees free access of the trypsin during the subsequent disaggregating step. Some residual medium is aspirated with a Pasteur pipet. The follicles are covered by a minimal volume of 0.1% trypsin-EDTA solution (a droplet of the number of follicles is less than 5 mL for 50 or more follicles). The follicles are then incubated at 37° C. until detachment of the outermost ORS cells becomes visible. This detachment procedure usually takes approximately 15-20 minutes, but its completeness has to be checked under the inverted microscope. Effective trypsinization is recognized by the fact that the ORS tissue becomes loosened and single ORS cells are visible around the follicle.

The trypsin is inactivated by the addition of five times of completed culture medium (5 mL if 1 mL trypsin was used). The follicles are pipetted up and down through a Pasteur pipet several times, taking care to avoid the formation of foam. The cell suspension still containing the follicles is then transferred into a 50-mL tube. The 35-mm dish is rinsed twice with 1 mL of culture medium, which is added to the 50-mL tube. The medium in the 50-mL tube is made up to 7 mL. Further release of ORS cells still adhering to the follicles is achieved by vigorous pipeting of the suspension through a 5-mL pipet at least 50 times. The suspension is then diluted with culture medium in such a way that the final volume in milliliters corresponds to the number of follicles prepared. If only few follicles are prepared, it is better to reduce the volumes during the isolation procedure in order to avoid a centrifugation step.

Primary Cultivation of ORS Cells

The ORS cell suspension is distributed in culture dishes containing a preformed feeder layer (3T3 or fibroblasts) and the hair follicles mostly denuded from the ORS tissue are removed with fine tweezers.

The cultures are incubated at 37° C. in air with 5% CO₂. The ORS cells from 1 follicle were cultured in a one ml culture medium in a 10-cm² culture disk. A seeding density of about 1×10³ cells/cm² is achieved, so that only few round cells are visible over the feeder layer. Spreading of ORS cells occurs only after 24 days, with the ORS cells being located predominantly between feeder cells. At this time, the ORS cells display the typical epitheloid morphology, with a well-discernible nucleus and a large cytoplasm. With time, colonies of ORS cells develop, which push aside the feeder cells. After four days, one ml of the fresh medium was added into the culture dish. The first medium change is done not before culturing for 7 days.

Thereafter, the medium is changed three times a week. The medium changes remove the detached feeder cells. As the size of the colonies increases, the ORS cells become more compactly arranged, while their apparent size decreases and the cytoplasm becomes less striking. During the first culture days, the proliferation seems rather slow, but the cell number increases rapidly as soon as the culture is in the logarithmic growth phase. Around days 12-14, the culture is 80% to 100% confluent.

Subcultivation of ORS Cells

For subcultivation of the ORS cells, residual feeder cells are first selectively removed by incubation at 37° C. for 2-3 minutes with 0.02% EDTA in PBS without calcium and magnesium. Effective removal of the feeder cells is obtained by vigorously pipeting the EDTA solution several times against the feeder cells. The EDTA solution is then aspirated, and the ORS cells rinsed three times with PBS without calcium and magnesium to remove all the feeder cells. The ORS cells are incubated at 37° C. in 0.1% trypsin-0.02% EDTA in PBS without calcium and magnesium, for example, a 0.5 mL/35-mm dish. Cell disaggregation is usually completed within 8-10 minutes, but has to be checked under the inverted microscope. Trypsin is blocked by adding 1.5 mL of culture medium per 35-mm culture dish. A single cell suspension is obtained by vigorous pipeting through a 5-mL pipette. A cell count is performed using, for example, a hematocvtometer chamber. The cells are centrifuged at 250 g for 8 minutes at room temperature. The supernatant is aspirated and the cells are resuspended in the selected media.

Secondary cultures of ORS cells are best performed in low calcium media on tissue-culture plastic in the presence of feeder cells. In this case, plating densities as low as 1.0×10⁴ cells/cm² are easily achievable, with a maximal number of subcultures around 3-5. The subculture of ORS cells could be continued based on the requirements of the ORS cells for the preparation of the graft. ORS cells after amplified were stored at −80° C. for seeding on the supporting membrane.

Alternative Culture Procedure to Achieve ORS Cell Amplification

One alternative procedure used to produce and expand keratinocytes is to isolate it from the culturing of whole hair follicles which were explanted into the collagen gel in a culture dish. Human hair follicles are removed from the scalp using a sterile technique and immersed in DMEM with antibiotics. The remainder of the sebaceous gland duct represents the upper limit of the extracted tissue so that the outer root sheath portion at the level of the infundibulum is always lacking. The bulbs were removed with scissors, since their soft end would hamper the implantation of the explant into the collagen gel. Hair follicles are cut into pieces (usually two) in order to reduce the size of the explant to ensure that it would maintain an upright position.

The explants are implanted into the fresh collagen gel in an upright position. A limited portion of the explant is actually inserted into the collagen gel. Five, half-follicles are implanted/35-mm dishes. The explants are incubated in the Human Hair Follicle Cell Primary Culture Medium (ORSCM) in a humidified incubator at 37° C. using 5% CO₂. Keratinocytes from the implanted hairs usually reach 70% to 80% confluency after 8-14 days in culture. The culture can be maintained for at least two months. Subculturing of ORS cells is described in the prior section.

Cultivation of Epithelial Cells from Other Lining Epithelia

Oral Epithelium

Human oral mucosa is composed of different types of epithelium traditionally classified as lining (noncornified epithelia), masticatory (orthokeratinising or parakeratinisiing cornified epithelia) and specialized types (dorsal part of the tongue). Gingival mucosa (masticatory epithelium) is similar to the epidermis both in terms of morphology and biochemistry. The epithelium can be cultured for providing seeding cells to construct a graft for the treatment of oral mucosa defects or skin defects.

Urethral Epithelium

Urethral epithelium is isolated from urethral meatus in a biopsy. The epithelial cells are cultured as seeding cells for reconstruction of urethral mucosa equivalent to treat congenital defects such as hypospadias.

Corneal Epithelium

The anterior ocular surface is covered with the highly specialized conjunctival and corneal epithelia. The conjunctive epithelium is well vascularised and consists of loosely organized cell layers populated by mucin-secreting goblet cells. The corneal epithelium is a stratified squamous epithelium, devoid of goblet cells, as well as of other cell types, with a cuboid basal layer lying on the avascular corneal stroma. Visual acuity is dependent on the corneal epithelium, the integrity of which is maintained by the centripetal migration of stem cell-derived transient amplifying cells. Surgical removal of the limbal region results in collection of corneal epithelium for culture and reconstruction of corneal equivalent graft for treatment of corneal diseases or injury.

VIII. Construction of Skin Equivalents

Construction of epidermal layer

One prepared insert with supporting membrane, amnion or chorion, are placed in the upright chamber A side of the culture dish. The supporting membrane is a de-epithelium or de-trophoblast ready to seed the skin cells. The expanded population of keratinocytes derived from primary cell cultures of the epidermal cells prepared from skin or hair follicle cells were mixed with dermal cells in equal ratios. The mixed cell suspension is seeded on the side of basement membrane of amnion or chorion in chamber A. The epidermal cell primary culture medium is added into the inside of chamber A along with the culture dish. For the first three days of culture a mixture of conditioned media and fresh media is added to the cultures. After the first three days of growth the culture medium is replaced every three days. The formation of epidermal equivalent on the supporting membrane generally requires ten to fourteen days to be completed.

Construction of Dermal Layer

Following seven to ten days of growth the insert carrying the epidermal cells on the supporting membrane is reversibly turned (180°) using sterile forceps, to make chamber B face in the upright position. Frozen dermal cells are thawed and seeded on the connective tissue side of the supporting membrane in chamber B. The same medium used as the epidermal cell culture is added in chamber B. After a three to four day cell culture, the insert is reversed using a sterile forceps so that chamber A is right side up and the cells cultured for an additional five to seven days. At this point, the skin equivalent graft is formed and ready for transplantation into wounded skin.

Construction of Different Skin Equivalents Based on the Cellular Sources

Autologous graft: Epidermal cells are derived from the recipient's skin or hair follicles and dermal cells from recipient's connective tissue.

Semi-autologous graft: Epidermal cells are derived from the recipient's hair follicles and dermal cells from allogenic connective tissue.

Allogenic Graft: Both epidermal cells and dermal cells are derived from allogenic sources.

Fibroblast graft: The graft is only composed of fibroblasts on the supporting membrane.

Construction of Other Tissue Equivalents

Human oral mucosa equivalents: Human oral mucosa cells are used as the seeding cells in the insert for construction of human oral mucosa equivalent graft.

Human urethral mucosa equivalent: Human urethral mucosa cells are used as the seeding cells in the insert for construction of human urethral mucosa equivalent graft.

Human corneal equivalent: Human corneal cells are used as the seeding cells in the insert for construction of human corneal equivalent graft.

IX. Detections of Growth Potential of Keratinocytes on the Grafts

The keratinocyte clones are formed in the primary epidermal cell cultures using epidermal cell primary culture medium. The colonies are large with 1-2×10⁵ cells per colony after 14 days of culture, and a very smooth and regular perimeter formed by migrating involucrine-negative cells. When subcultivated, clones generate large and smooth daughter colonies with an efficiency of 100%. A cell giving rise to a called holoclone can generate as many as 1×10⁴⁰ progeny (over 140 doublings), i.e. enough epithelium to cover several times an adult human body, whose epidermis contains approximately 8×10¹⁰. Therefore, the basal keratinocyte generating a holoclone has the essential characteristic to be considered as a stem cell, namely a tremendous potential for proliferative self-renewal. The following detection systems are used for detection of cell viability, re-proliferation, and stem-like cell marking (Y. Barrandon et al. Cell Size As A Determinant Of The Clone-Forming Ability Of Human Keratinocytes. Proc Natl Acad Sci USA. 1985, 82(16):5390-4.; Y Barrandon, et al. Three Clonal Types Of Keratinocyte With Different Capacities For Multiplication. Proc Natl Acad Sci USA. 1987, 84(8):2302-6.; M. B. Mathor et al. Clonal Analysis Of Stably Transduced Human Epidermal Stem Cells In Culture. Proc Natl Acad Sci USA. 1996, 93(19):10371-6.; A. Rochat et al. Location Of Stem Cells Of Human Hair Follicles By Clonal Analysis. Cell. 1994, 76(6):1063-73.; Barrandon and Green, 1985, 1987; Mathor et al, 1996; Rochat et al., 1994).

Detection of Integrin Beta-1 Positive Cells

Integrin beta-1 has been considered as a marker of epidermal stem cell (F. M. Watt. Stem Cell Fate And Patterning In Mammalian Epidermis. Curr Opin Genet Dev 2001, 11(4):410-7.; F. M. Watt. Role Of Integrins In Regulating Epidermal Adhesion, Growth And Differentiation. EMBO J 2002, 21(15):3919-26.; C. Bagutti et al. Dermal Fibroblast-Derived Growth Factors Restore The Ability Of Beta(1) Integrin-Deficient Embryonal Stem Cells To Differentiate into Keratinocytes. Dev Biol 2001, 231(2):321-33.). In present invention, anti-integrin beta-1 antibody (from Upstate Biotechnology) is used as a detecting marker of integrin beta-1 on the epidermal stem cells. The keratinocytes used for construction of the graft are cultured in a slide chamber. When the cells are touched and grown on the slide for about three days, the cells are then washed three times for 15 minutes with PBS, and then, fixed using 95% ethanol/5% acetic acid for one minute at 4° C. The fixed cells are incubated with 1% bovine serial albumin for 1 to 2 hours at room temperature and washed twice with PBS for 15 minutes. The antibody against human integrin beta-1 is diluted into 10·mu·g/ml in 1% bovine serial albumin and incubated overnight at 4° C. After washing three-times with PBS, the cells on the slide are incubated with anti-mouse IgG fluorescein conjugated secondary antibody in 1% bovine serial albumin for 1.5 hours at room temperature, and washed three times with PBS for 15 minutes. The positive cells are detected under a fluorescent microscope (Watt, 1998, 2001, 2002; Bagutti et al., 2001; Cotsarelis et al., 1999).

An alternative-detecting marker of epidermal stem cells using anti-cytokeratin 19 antibody.

Cytokeratin 19 has also been considered as a marker of epidermal stem cells (M. Michel et al. Keratin 19 As a Biochemical Marker Of Skin Cells In Vivo And In Vitro: Keratin 19 Expressing Cells Are Differentially Localized In Function Of Anatomic Sites, And Their Number Varies With Donor Age And Culture Stage. J Cell Sci. 1996. 109:1017˜1028.). In the present invention, cytokeratin 19 antibody (from DAKO) is used as a detecting marker of cytokeratin 19 in the epidermal stem cells. The keratinocytes used for construction of the graft are cultured in a slide chamber. When the cells are touched and grown on the slide for about three days, the cells are washed three times for 15 minutes with PBS, and then, fixed using 4% paraformaldehyde for fifteen minutes at 4° C. and washed two times with PBS for 15 minutes. The fixed cells are incubated with 0.1% saponins solution for 1 to 2 hours at room temperature and incubated with antibody against human cytokeratin 19 overnight at 4° C. After washing three-times with PBS, the cells on the slide are incubated with anti-mouse IgG fluorescein conjugated secondary antibody in 1% bovine serial albumin for 1.5 hours at room temperature, and washed three times with PBS for 15 minutes. The positive cells are detected under a fluorescent microscope.

Detection of Cell Viability using Trypan Blue:

The pure keratinocytes from the culture insert are digested using 0.25%-trypsin-EDTA solution. The cells are stained by 0.04% trypan blue (from Sigma) and total numbers of cells are counted under a microscope. The ratio of un-stained and stained cells is calculated based on the number of the un-stained and stained cells. The results from the present invention indicated that the living cells (un-stained cells) are about 85% (80% to 95%) (as shown in Table 1)

TABLE 1
Viability Tests Using Trypan Blue Stain

| Insert | | | |
|---|---|---|---|
| No. | Keratinocytes Counted | Stained Cells | % (Dead Cells/Liv. Cells) |
| 168 | 250 | 27 | 10.8 |
| 169 | 310 | 35 | 11.2 |
| 176 | 300 | 28 | 9.3 |
| 177 | 300 | 31 | 10.3 |
| 178 | 300 | 29 | 9.6 |
| 179 | 300 | 36 | 12.0 |

Karyotype Analysis of the Cells from the Graft Successful preparations of epidermal cells and dermal cells have normal karyotypes. Both XX and XY cells will be derived. To determine whether the cells used in the graft exhibited normal karyotype, the cells which are cultured as described herein are tested. Approximately 10-20 metaphase stage karyotypes from the prepared cells are tested by examining the cell's chromosomes for both structural and numerical abnormalities. Before seeding the cells onto supporting membrane, the cells from human hair follicles are karyotyped and are normal 46,XY and 46,XX, respectively.

When the cells reached 40-50% confluence in the culture dish, 0.02·mu·g/ml colcemid (GIBCO BRL) is added to the culture medium and the cells are continuously cultured overnight at 37° C. in 5% CO₂, 95% air. The Cells are subsequently washed in PBS, treated with 0.25% trypsin-EDTA for 10-15 minutes at 37° C., and removed and centrifuged for five minutes at 800·times·gravity. The Cells are fixed for five minutes in cold Camoy's fixative (3:1 volume of absolute methanol to glacial acetic acid), washed in PBS, centrifuged as above, and resuspended in 0.5 ml of Carnoy's fixative. A pipette drop of the resulting cell suspension is transferred onto microscopic slides that are perished with Camoy's fixative. The Slides are air-dried, Giemsa stained (GIBCO, BRL) and rinsed with tap water. After a second drying, the slides are cover slipped and viewed under oil immersion using light microscopy at 400× magnification.

The keratinocytes and fibroblasts examined had a normal complement of human chromosomes (i.e., 44 autosomes and 2 sex chromosomes). Additionally, no breaks, deletions, additions or other abnormalities in the shape or number of chromosomes are observed.

X. Transportation Delivery of Skin Equivalents

Two methods are utilized to transport and/or ship the graft in the present invention. In the first method, When the graft is ready to be used for transplantation, the insert containing the graft is placed into a sterile container, filled with culture medium and sealed with Parafilm. This method is suitable for shipment at 4° C.-10° C. To minimize vibrations and shaking which can damage the membrane system during transportation, the container should be such a size as to just allow the insert to fit into the transportation container when filled with media. Upon delivery of the transported insert and graft, the insert and cells are re-cultured with the same culture medium as a fresh culture dish for an additional one to two days. At this point the graft is ready for use.

The second method for transportation and shipping of the graft in the present invention requires immersion of the harvested skin equivalent or graft in a cryoprotectant solution for a period of time sufficient to completely perfuse the sample, preferably between one and two hours, but most preferably, for about one hour. This method of cryopreservation allows the graft to be slowly frozen and stored at or below −70° C. Tissues cryopreserved by this method are stable to fluctuations in temperature between −70° C. to −196° C. Short term storage is possible at −76° C., the temperature of dry ice, for transport and shipping. It is preferable to freeze tissues to a temperature at or below −120° C., the glass transition temperature of water. It is more preferable to freeze tissues to a temperature at or below −140° C., a temperature approaching the temperature of a liquid nitrogen. It is most preferable to freeze tissues to a temperature at or below −196·degree·C., the temperature of liquid nitrogen. The cryoprotectant solution used in present invention contains 2M Glycerol in DMEM.

The frozen cryopreserved cultured graft is thawed by rapidly warming such that the tissue is thawed in about from 1 to 3 minutes. Suitable methods for warming frozen harvested graft at a high thawing rate, include warming using a water bath or warming using induction heating. Preferably, the frozen graft is thawed by direct addition of the culture media, warmed to 37° C., to the surface of the cryopreserved graft.

Due to the toxic nature of the cryoprotectant agents, the cryoprotectant solution is removed from the thawed graft within about 15 minutes after thawing, preferably as soon as possible after thawing, to avoid damaging the viability of the tissue or tissue equivalent. Once the graft is thawed, the cryoprotectant solution is replaced with a culture medium at a physiological pH (about 6.8 to 7.4 pH).

XI. Grafting Procedure and Clinical Uses

Grafting Procedure

Male SD rats weighing approximately 250-300 g are anesthetized with sodium phenobarbital. A grafting bed approximately the same size as the graft with rat's skin equivalent is prepared by removing the full thickness of the skin from the back of each animal. The graft is placed and stitched at the border of the graft with the skin border of the wound. The wound is then covered with a vaseline-impregnated Telfa pad and a Telfa pad soaked in Earle's salt Solution. These bandages are covered by wrapping the body with several layers of Elastoplast. At time intervals ranging from nine days to thirteen months after the graft is originally applied, the animals are again anesthetized and the entire graft is excised. Half of the graft is fixed in phosphate-buffered formalin, dehydrated in ethanol, and embedded in paraffin. A central portion of the other half of the graft is trimmed of underlying fat tissue, cut into 2-3 mm³ pieces and placed in tissue culture to allow the resident fibroblasts to grow out.

Clinical Uses of the Skin Grafts

The skin-equivalent graft described herein is suitable for treatment of a wound or defect of the skin of a human being or other mammal. It is particularly suitable for skin injury or ulcers such as massive burns.

Use in the treatment of burns. The place of the graft in management of burns can be considered with respect to donor sites, partial-thickness of the injury, and full-thickness burns.

Use in the treatment of Leg Ulcers. The majority of leg ulcers are of venous origin. The wound needs to be prepared before grafting the skin equivalent.

Use in the treatment of Traumatic and Chronic Ulcers of Skin. The skin equivalents prepared in the invention can be used to repair the wound prominently.

Use in the treatment of latrogenic Wounds and Ulcers. Elective procedures in which a raw surface is either routinely or occasionally created are diverse. In some of these, the surface is routinely left to granulate and epithelialize spontaneously (for instance, following the excision of a pilonidal sinus), whereas in others it may be deemed apposite to delay-graft. The skin equivalent graft can be provided in both of these circumstances, either to achieve more rapid healing or provide an uncontaminated surface upon which a skin graft will readily take.

Use in the treatment of any surgical defects of the skin.

Use in gynecological applications. The skin equivalent graft prepared by present invention can be used for construction of an artificial vagina, and repair of the vagina defects.

Use in the treatment of Vitiligo. It has been shown that the skin equivalent prepared in the present invention contains melanocytes which proliferate rapidly in the same culture conditions that allow keratinocytes growth. Patients suffering from vitiligo are so inten[t]sely desirous of treatments that they accept these long-lasting therapies.

XII. Industrial Production

Those skilled in the art will recognize, or be able to ascertain using no more than routine experimentation, other equivalents for the specific reactants, catalysts, steps, techniques, etc., described herein. Such equivalents are intended to be included within the scope of the following claims.

I claim:

1. A method of preparing a transplantable graft of an engineered tissue equivalent for treatment of body tissue defects, comprising:
a) preparing an assemblable culture insert for producing a transplantable graft with living cells; said assemblable culture insert comprising:
i) a female slip slave vessel having holes in its bottom wall to create a pathway for cell culture medium exchange between outside and inside a lower chamber of said vessel when the insert is placed in a cell culture container;
ii) a male slip ring vessel insertable in said female slip slave vessel with a supporting membrane to create an upper inner chamber; and
iii) a sheet of an extraembryonic biological supporting membrane that creates said upper inner chamber and lower chamber when mounted and stretched between the intact inserted assembly of the slip slave vessel and the male slip ring vessel;
b) preparing seed cells of main functional cells and stromal cells;
c) constructing a tissue equivalent graft of a histological configuration of mammalian tissue through seeding and growing living cells on said extraembryonic biological supporting membrane; and
d) disassembling said culture insert and releasing the tissue equivalent graft for treatment of body tissue defects.

2. The method of claim 1 wherein said tissue equivalent graft includes any one of:
a) skin equivalent;
b) mucosal equivalent;
c) corneal equivalent; and
d) other body membrane equivalents.

3. The method of claim 1 wherein said tissue equivalent graft is employed for any one of:
a) repairing skin defects;
b) reconstructing mucosal surfaces for treatment of mucosal defects;
c) constructing ocular surface for treatment of ocular surface defects;
d) healing of corneal epithelial defects; and
e) repairing any body membrane defects.

4. The method of claim 1 wherein said transplantable graft is an engineered skin substitute for treatment of skin defects.

5. The method of claim 4 wherein said engineered skin substitute is an equivalent structure of skin having a surface of epidermal cell layer and an underline dermal layer including connective cells and tissue matrix.

6. The method of claim 4 wherein said engineered skin substitute is one of:
a) autologous graft comprising said biological supporting membrane and autologous epidermal and dermal cells;
b) semi-autologous graft comprising said biological supporting membrane and autologous epidermal cells and allogenic dermal cells;
c) allogenic graft comprising said biological supporting membrane and allogenic epidermal and dermal cells;
d) fibroblast graft comprising said biological supporting membrane and fibroblasts;

7. The method of claim 5 wherein said epidermal cell are living keratinocyte derived from epidermis of skin.

8. The method of claim 5 wherein said epidermal cell are living keratinocyte derived from hair follicle.

9. The method of claim 5 wherein said connective cells are living fibroblasts derived from dermis of skin.

10. The method of claim 5 wherein said tissue matrix is composed of connective fibers.

11. The method of claim 4 wherein said skin defects are skin ulcers caused by different chronic diseases.

12. The method of claim 4 wherein said skin defects are skin wounds caused by different skin injuries.

13. The method of claim 4 wherein said skin defects are caused by surgery.

14. The method of claim 1 wherein said extraembroynic biological supporting membrane is a de-epithelial amnion, and has a layer of basement membrane and layers of connective tissue.

15. The method of claim 1 wherein said extraembroynic biological supporting membrane is a de-traphoblast chorion, and has a layer of pseudo-basement membrane and layers of connective tissue.

16. The method of claim 1 wherein said cell culture container is a cell culture dish or a cell culture plate

17. The method of claim 1 wherein said construction of the graft comprises:
a) assembling the insert and placing the insert in said cell culture container;
b) removing the epithelium and exposing the basement membrane of amnion;
c) seeding the living cells on the basement membrane side and connective tissue side through chambers of the insert;
d) Growing the cells on the membrane with culture medium; and
e) disassembling the insert and releasing the transplantable graft.

18. The method of claim 1 wherein construction of the graft comprises:
c) assembling the insert and placing the insert in said cell culture container;
d) removing the traphoblast layer and exposing the pseudo-basement membrane of chorion;
c) seeding the living cells on the basement membrane side and connective tissue side through chambers of the insert;
d) growing the cells on the membrane with culture medium; and
e) disassembling the insert and releasing the transplantable graft.

19. The method of claim 1 wherein growing living cells is enhanced on/in a biological supporting membrane, by:
a) forming an acidic solution of collagen;
b) adding the solution to a chamber of the insert and raising the solution to a pH level sufficient to precipitate collagen onto said extraembryonic biological supporting membrane;
c) incubating the solution with the membrane in the insert before seeding cells; and
d) forming a cell culture medium with multiple factors.

20. A method of inhibiting cell differentiation of keratinocytes, comprising:
a) forming a keratinocyte culture medium with a low concentration of calcium; and
b) culturing the keratinocytes with the medium.
